(19) United States
(12) Patent Application Publication
(10) Pub. No.: US 2025/0057860 A1
(43) Pub. Date: Feb. 20, 2025

(54) READY-TO-DILUTE FORMULATION

(71) Applicant: EMPHASCIENCE, INC., Pittsford, NY (US)

(72) Inventor: Rebanta BANDYOPADHYAY, Webster, NY (US)

(21) Appl. No.: 18/705,190

(22) PCT Filed: Oct. 25, 2022
(86) PCT No.: PCT/US2022/078626

§ 371 Date: Apr. 26, 2024

Related U.S. Application Data
(60) Provisional application No. 63/263,044, filed on Oct. 26, 2021.

(51) Int. Cl. A61K 31/675; A61K 9/00; A61K 47/10; A61K 47/26; A61K 47/32; A61K 47/34

(52) U.S. Cl. CPC A61K 31/675; A61K 9/0019; A61K 47/10; A61K 47/26; A61K 47/32; A61K 47/34

(57) ABSTRACT

A ready-to-dilute formulation containing 0.5-5 wt. % of a nucleoside or nucleotide pro-drug; 2-15 wt. % pyrrolidone-containing compound; and 65-97.5 wt. % of one or more co-solvent(s) is described, particularly wherein the pyrrolidone-containing compound is polyvinylpyrrolidone and the nucleoside or nucleotide pro-drug is remdesivir. An injectable solution containing the ready-to-dilute formulation and an aqueous diluent is also described, along with methods of treating viral diseases using the injectable solution.

### TECHNICAL FIELD

The present disclosure relates to ready-to-dilute formulations, injectable solutions and methods of treating viral infections for the effective treatment of viral infections in humans and other animal species caused by viruses, in particular, RNA viruses.

### BACKGROUND

Viral infections can have a detrimental effect on life. Not only can there be an immediate impact on the health of an infected individual, the contagious nature of some viral infections can also have far reaching effects on the functioning of communities, businesses, services and the overall economy. This has been illustrated, for example, by the novel viral infection, COVID-19, which surfaced in 2019 resulting in a worldwide pandemic. COVID-19 presents the risk of severe respiratory failure and death in some patients. In many cases, the progression to acute symptoms occurs in older patients and in those with underlying medical conditions such as hypertension or diabetes.

Coronaviruses, in particular, are enveloped RNA viruses with a positive-sense, single-stranded RNA genome that infect both animals and humans. Diseases from coronavirus include the common cold, severe acute respiratory syndrome (SARS), Middle East respiratory syndrome (MERS), and severe acute respiratory syndrome by coronavirus 2 (SARS-COV-2), the causative pathogen of the disease commonly known as COVID-19. The rapid emergence and spread of such viral infections allow little time for the development of vaccines. In the absence of clinically effective and safe vaccines, widespread immunization and controlling the virus becomes almost impossible. Even with clinically effective and safe vaccines, vaccine uptake in the population is unlikely to reach 100%. Breakthrough infections can also occur after vaccination, especially for the immunocompromised. Therefore, alongside the development and administration of vaccines, there is also a need for the effective treatment of viral diseases such as COVID-19.

Phosphoramidate, phosphonamidate, phosphoester or sugar-ester prodrugs of many nucleotide or nucleotide analogs are designed to have better membrane permeability and higher cellular uptake to elicit better antiviral activity. These antiviral drugs commonly carry a single phosphate moiety (monophosphate) attached to the parent nucleoside along with other ‘protecting groups’. The monophosphate group is a precursor to the formation of the active nucleoside triphosphate metabolite inside the cell and helps bypass the rate limiting first phosphorylation step. To promote cellular uptake, the monophosphate moiety is ‘protected’ with esters formed by ‘protecting groups’, such as with phenols and amino acids, which mask the negative charge of the phosphate group and help improve passive diffusion rate through the cellular membrane. However, even before entering the cells, the breakdown of the prodrug is sometimes initiated even in contact with aqueous media and physiological fluids and, especially by esterases that cleave the ester and expose the parent nucleotides and nucleosides that then serve as precursors to the active triphosphate analog. Other sugar-ester nucleoside or nucleotide pro-drugs comprise an ester group on the 5-OH, 3-OH, or 2-OH of the nucleoside, wherein the ester is hydrolysed to the nucleoside in vivo.

The monophosphate nucleotide prodrug remdesivir (also known as GS-5734), has been approved for use against COVID-19, while also demonstrating anti-viral properties against a range of other viral infections. Remdesivir is a lipophilic adenosine monophosphate analog pro-drug that is converted into the active triphosphate (GS-443902) inside the cell. By design, as with other phosphoramidate or phosphonamidate prodrugs, the prodrug remdesivir is designed to undergo rapid hydrolysis and de-esterification following administration to convert to the active nucleoside metabolite, GS-441524, and to the triphosphorylated active, GS-443902, inside the cell.

The currently marketed pharmaceutical compositions of remdesivir are formulated either as a lyophilized powder or a concentrated, ready-to-dilute solution of remdesivir (see further details of the dosage forms below). Both of these formulations must be diluted ahead of injection under aseptic conditions and used within 2 days.

The recommended dosage for adults and pediatric patients 12 years of age and older and weighing at least 40 kg is a single loading dose of Veklury® 200 mg on Day 1 via intravenous infusion followed by once-daily maintenance doses of 100 mg from Day 2 via intravenous infusion up to a maximum duration of 10 days. Veklury® is administered over 30 to 120 min by IV Infusion only.

The two available dosage forms are as follows:
a. Veklury® for injection.
   Supplied as 100 mg lyophilized powder in vial that needs to be reconstituted with sterile Water for Injection prior to diluting in a 100 mL or 250 mL 0.9% sodium chloride (normal saline or NS) infusion bag

Lyophilization protects the prodrug, remdesivir, from hydrolysis and premature degradation during manufacturing and in the course of storage and distribution, especially in the presence of moisture. However, lyophilization is expensive and requires the additional step of reconstitution of the lyophilized powder prior to further dilution, thus adding to the time and cost of administering the drug, and increasing the risk of loss of sterility. The lyophilized powder also contains 3g of sulfobutylether β-cyclodextrin (SBEBCD) for every 100 mg of the prodrug as a complexing and solubilization agent which is used to increase the solubility of remdesivir to assist in reconstitution and dilution prior to administration. Since remdesivir is insoluble in water, the presence of a solubilizing aid, such as SBEBCD, is considered critical to achieve even minimal concentrations of ca. 1 mg/mL in the infusion medium.
b. Veklury® injection
   Supplied as a concentrated solution, 100 mg/20mL [5 mg/mL] solution in vial that must be diluted in a 250 mL normal saline infusion bag.

The concentrated solution (5 mg/mL remdesivir) product contains even higher quantities of SBEBCD (6g for every 100 mg of remdesivir) for solubilization and can only be stored or transported at 2-8° C., requiring cold chain transportation/shipping and refrigerated storage. Unopened vials of the product can have a shelf-life of only around 12 months under refrigeration. Prior to opening or breaking the seal, vials of the concentrated solution can be stored up to 12 hr at room temperature for thawing and before dilution. After opening and dilution, the final, diluted solution is only stable for 48 hr at refrigerated temperature (2-8° C.) or 24 hr at room temperature. Even though the concentrated solution reduces the number of steps required to dilute the product prior to infusion, it is much more susceptible to degradation and requires refrigerated storage. It also contains double the quantity of the solubilizing aid, i.e. 6 g SBEBCD for every 100 mg of remdesivir. Hence, a higher dilution with 250 ml of 0.9% sodium chloride to a final concentration of 0.4 mg/ml to 0.8 mg/ml of remdesivir (and 24 mg/mL to 48 mg/mL SBEBCD) helps ensure that the final solution is isotonic.

In view of the above, it is clear that there are a number of challenges for manufacturing, storing and administering Veklury® due to thepoor solubility and stability of remdesivir. In addition, both dosage forms and formulations include high amounts sulfobutylether β-cyclodextrin (SBEβCD) as a complexing and solubilizing excipient at up to 30 to 60 times the weight of the remdesivir dose. Hence, up to 12 g of SBEBCD can be administered along with the 200 mg maximum unit dose of remdesivir by intravenous infusion. This is disadvantageous because following intravenous administration, cyclodextrins are excreted intact by renal excretion and can accumulate in the kidney at high doses. This can cause vacuolation of the tubular cells in the kidney leading to possible renal impairment. In children, and in patients with lower glomerular filtration rate due to impaired kidneys, high doses of SBE/CD can result in osmotic nephrosis and extra renal adverse effects due to higher blood levels of SBE/CD and increase in osmotic pressure. As a result, the product label for Veklury® recommends that only those patients with properly functioning kidneys and an estimated glomerular filtration rate of at least 30 min/min should be considered for treatment with Veklury®. Furthermore, to match the tonicity of blood despite the high quantities of SBEBCD in the product and to avoid precipitation due to the lack of solubility of remdesivir, the product can only be diluted with 100 to 250 ml of normal saline (NS) for a final concentration range of 0.4 to 1.25 mg/mL, and should not be mixed with other infusion media or injections. For acutely ill patients with hemodynamic imbalance, excess sodium intake may sometimes be contraindicated. Although the lyophilized powder is stable at room temperature, lyophilization is an expensive manufacturing step and the powder requires two step reconstitution and dilution prior to administration, thus increasing the time and cost required for administering the drug, and the risk of loss of sterility. The concentration solution has a limited shelf life, requires refrigerated storage and cold-chain shipping/transportation that all add to its limitations, even though it requires one less step of dilution prior to administration.

For many nucleotide or nucleoside prodrugs, including remdesivir, there is therefore a need to develop novel and improved injections and/or ready-to-dilute injection formulations that are stable and do not contain large amounts of cyclodextrin and cyclodextrin derivatives or other solubilizing aids. Designing stable injectable formulations for such nucleotide or nucleoside prodrugs, including remdesivir, that are susceptible to rapid hydrolysis/de-esterification in solution or in contact with water and physiological fluids is especially challenging. Such improved formulations should preferably have good solubility to form a concentrated solution dosage form that can contain the entire dose in a relatively small volume of liquid solution phase and result in lower cost due to lower volume-fill, lower raw material, packaging and shipping costs. In addition, these concentrated solutions should be capable of being diluted with various standard IV infusion media (not just normal saline) at different final concentrations of the drug, to ensure optimum composition for the infusion for all types of patients, and for the ease of administration when some IV infusion fluids are in short supply. While poorly soluble and unstable drugs have sometimes been formulated as suspensions to avoid the use of solubilizing aids or to reduce the extent of degradation in the solution phase, suspension dosage forms are susceptible to the risk of settling during storage, un-uniform mixing, and lack of dose uniformity. Suspension dosage forms pose significant challenges to sterilization, as well. There is therefore a need to provide novel and improved solution dosage formulations, that are very stable and which can be steam-sterilized or terminally filtered to eliminate the need for expensive manufacturing methods, including lyophilization. Such a formulation should also be amenable to dilution in sterile water-for-injection (sWFI) or various other IV infusion media prior to administration as an infusion. Hence, novel and improved ready-to-dilute formulations that have good chemical stability, have an extended shelf-life (≥12 months), and/or can be stored and transported without refrigeration, can lower cost, extend flexibility of use in various patients and under different circumstances, and generally augment therapeutic use.

### SUMMARY OF INVENTION

In a first aspect of the invention, there is provided a ready-to-dilute formulation comprising
0.5-5 wt. % of a nucleoside or nucleotide pro-drug, wherein the nucleoside or nucleotide pro-drug is a phosphoramidate, phosphonamidate or phosphoester or sugar-ester nucleoside or nucleotide prodrug;
2-15 wt. % a pyrrolidone-containing compound; and
65-97.5 wt. % of one or more co-solvent(s).

In a preferred embodiment, the nucleotide or nucleotide pro-drug is remdesivir and/or the pyrrolidone-containing compound is polyvinylpyrrolidone.

In some embodiments, the formulation is a suspension or a solution of the nucleotide or nucleoside pro-drug. In preferred embodiments, the formulation is substantially free from water, i.e., defined as comprising less than 1 wt. %, or less than 0.5 wt. %, or less than 0.1 wt. % water. This protects the pro-drug from undergoing significant hydrolysis, de-esterification and/or degradation during manufacturing and storage leading to unacceptable levels of loss of the pro-drug. In some embodiments, the ready-to-dilute formulation is a concentrated solution comprising at least 10 mg/ml, or at least 15 mg/ml of the nucleotide or nucleoside pro-drug, (e.g. remdesivir), such that prior to administration to a patient, it can be diluted with aqueous media containing water or an aqueous mixture of the pro-drug. In some embodiments, the aqueous mixture is a solution. In other embodiments, the aqueous mixture is a nanosuspension. In a preferred embodiment, the water-based mixture is suitable for direct administration. The pyrrolidone-containing compound is a good H-bond acceptor. Thus, in a variation of the first aspect, the pyrrolidone-containing compound may be replaced by a different H-bond acceptor.

In a second aspect of the invention, there is provided an injectable solution comprising the ready-to-dilute formulation of the first aspect, and an aqueous diluent. The aqueous diluent is suitable for intravenous (IV) infusion following mixing with the concentrated solution, e.g., wherein the aqueous diluent is of suitable pH and tonicity such that the final mixture following dilution of the concentrated solution is isotonic with blood and is suitable for administration as an intravenous (IV) infusion.

In a third aspect of the invention, there is provided a method of treating a viral infection, the method comprising intravenously administering to a subject in need thereof the injectable solution of the second aspect.

Also described herein in a fourth aspect, is a ready-to-dilute formulation comprising
0.5-5 wt. % remdesivir;
2-15 wt. % pyrrolidone-containing compound; and
65-97.5 wt. % of one or more co-solvent(s). In a preferred embodiment, the pyrrolidone-containing compound is polyvinylpyrrolidone.

Also described herein in a fifth aspect, is an injectable solution comprising the ready-to-dilute formulation of the first or fourth aspect, and an aqueous diluent or aqueous mixture. The aqueous diluent or aqueous mixture preferably comprises water-for-injection (WFI) or other hypotonic intravenous infusion media, including half normal saline (0.45% sodium chloride solution in water).

Also described herein, in a sixth aspect, is provided a solution comprising a nucleoside or nucleotide pro-drug, polyvinylpyrrolidone and PEG, wherein the nucleotide or nucleotide pro-drug is a phosphoramidate, phosphonamidate or phosphoester or sugar-ester pro-drug. In some examples, the nucleoside or nucleotide pro-drug is remdesivir. In some examples, the PEG is PEG 300 and/or PEG 400. In some examples, the nucleoside or nucleotide pro-drug is remdesivir and the PEG is PEG 300.

Also described herein, in a seventh aspect, is a pharmaceutical composition comprising the ready-to-dilute formulation of the first or fourth, or the solution of the sixth aspect, and optionally a pharmaceutically acceptable excipient.

Also disclosed herein, in an eight aspect, there is provided a method of copackaging the concentrated ready-to-dilute formulation and an aqueous diluent or aqueous mixture in the same primary packaging container, wherein the ready-to-dilute formulation is separated by a temporary barrier from the diluent. In some embodiments, the primary packaging container is a dual chambered intravenous infusion bag, and wherein the temporary barrier can be removed by a simple manipulation of the bag from the outside prior to administration and the two components can be mixed to form a diluted, infusion-ready solution without needing to physically transfer any volume of liquid to and from two separate primary packaging containers containing the dosage form and diluent. The separation of the concentrated solution and the diluent allows for maximum stability of the nucleoside or nucleotide pro-drug (e.g. remdesivir) while in storage and, at the same time, eliminates the need for aseptic transfer between containers and the risk of loss of sterility during dilution.

In the above aspects, even though the pharmaceutical formulation is being described as a ready-to-dilute formulation taking into account the need for dilution to match the osmolality (or tonicity or osmotic pressures) of physiological fluids for the purpose of direct administration as in case of an intravenous injection, such a formulation, in the form of a concentrated solution, may also be administered by other routes of administration without dilution when the osmolality of the solution is inconsequential. For example, the concentrated solution with or without added flavorants may be administered orally without dilution to deliver the effective dose of a nucleoside or nucleotide prodrug (i.e. the phosphoramidate, phosphonamidate, polyester or a sugar-based nucleoside or nucleotide pro-drug) in a relatively small volume of oral solution. In that aspect, the pharmaceutical composition may be an oral formulation (i.e. a pharmaceutical composition formulated for oral administration) or an intranasal formulation (i.e. a pharmaceutical composition formulated for intranasal administration).

Also described herein, in a ninth aspect, is a method of treating a viral infection, the method comprising administering to a subject in need thereof the pharmaceutical composition of the fifth aspect.

The present aspects of the invention or embodiments thereof have one or more of the following advantages:
The ready-to-dilute formulations described herein have high solubility of the nucleoside or nucleotide prodrug and extremely good chemical stability. For example, the prodrug (e.g. remdesivir) does not undergo de-esterification and/or hydrolysis or otherwise degrade or undergo chiral changes at controlled room temperatures (USP Controlled Room Temperature (CRT) 20 to 25° C.) over the course of its shelf-life, or even at elevated temperatures (30 to 40° C. to 50° C.) over short periods of time, and under conditions used for steam-sterilization (e.g. ˜ 121° C. for up to 30 minutes) in this ready-to-dilute solution. As a result:
   The ready-to-dilute formulations can be manufactured and stored easily under ambient conditions without limitations of environmental oxygen, UV light, and may be mixed at or exposed to elevated temperatures of up to 50° C. over short periods of time if necessary, for the purpose of increasing manufacturing efficiency or during shipping and, finally, even steam-sterilized or terminally filtered to render the solution sterile where necessary without impacting the potency and quality of the product.
   The ready-to-dilute formulations can have an extended shelf-life (e.g. at least 1 year, or at least 2 years, or at least 3 years) at 20° C. to 25° C., i.e. USP Controlled Room Temperature (CRT) storage.
   The ready-to-dilute formulations do not require refrigeration or cold-chain transportation/shipping.
   The ready-to-dilute formulations can be more easily manufactured and stored under ambient conditions (i.e. without special environmental controls such as low UV light or low oxygen).
   The ready-to-dilute formulations can be steam-sterilized by heating the product up to at least 121° C. for several minutes as is necessary to achieve adequate sterility.
   The ready-to-dilute formulations can be used to improve access to the medicine in places where cold-chain transportation/storage and aseptic processing/compound facilities are unavailable.
   The ready-to-dilute solution requires a single dilution step by directly transferring the dose to a container containing a suitable infusion medium comprising WFI or other acceptable IV infusion media containing substantial quantities of water and does not require reconstitution before dilution unlike any lyophilized product.
   In some aspects, the ready-to-dilute solution may be diluted in the same container containing a suitable diluent where the two solutions are temporarily separated by a removable barrier and such mixing can be undertaken without physically transferring the solution to the diluent and without the risk of exposure to a nonsterile atmosphere and loss of sterility.

In addition:
The components of the ready-to-dilute formulations are found to effectively solubilize the nucleotide or nucleoside prodrug (e.g. remdesivir) even when diluted with an aqueous diluent (e.g. an intravenous fluid medium), without the risk of immediate precipitation. Solubilization of remdesivir in the presence of water or an aqueous diluent, for example, is even further improved with the addition of a surfactant, such as polysorbate 80.
The ready-to-dilute formulations, solutions and injectable solutions described herein are free of cyclodextrins, including sulfyl-butyl ether β-cyclodextrin (SBEBCD). As a result, the formulations are less burdensome on patient's kidneys, and eliminate the safety risks with patients with poor kidney function and/or in younger patients.
As compared to Veklury® for injection which requires reconstituting a lyophilized powder, the ready-to-dilute formulation is pre-solubilized and can be diluted without reconstitution, thus reducing the risk of loss of sterility. It also facilitates the quick preparation of an injectable solution with reduced risk of adverse effects and inaccurate dosing.
The ready-to-dilute formulations can be readily diluted with various aqueous diluents (including sterile water for injection, half-normal saline or 5% dextrose solution) to obtain an injectable solution that is isotonic with blood. This provides increased flexibility as compared to Veklury® formulations, which can only be diluted with 0.9% sodium chloride solution.
The ready-to-dilute formulations and/or solutions described herein comprise a higher concentration of nucleoside or nucleotide pro-drug (e.g. remdesivir) as compared to Veklury® Injection, due to the solubilizing effect of the excipients, including one or more pyrrolidone-containing compounds and/or co-solvents on the pro-drug (e.g. remdesivir). As a result, the novel solution requires a lower fill-volume or dosage volume. The low fill-volume or dosage volume ensures that the entire dose can be filled into smaller vials of more conventional size (≤10 ml vials) than the 20mL fill-volume required for Veklury® Injection. The smaller fill volume and smaller vial size increases the speed of manufacturing, reduces the cost of goods and packaging, and reduces the cost of shipping and storage, as well as, allows the concentrated solution to be added into the diluent container without removing any quantity of the diluent to provide space for the concentrated solution to be added therein. Specifically, due to the higher concentration and lower dosage volumes, it is not necessary to withdraw and replace volume from the IV infusion fluid-bags/bottles before adding the drug to the infusion vial or bag prior to administration.
The novel ready-to-dilute solution eliminates the step of reconstitution in contrast to the lyophilized powder, and by reducing the number of aseptic steps, it lowers the risk of contamination and loss of sterility.
Due to effective solubilization of the nucleoside or nucleotide pro-drug (e.g. remdesivir), the compound can be present in the novel ready-to-dilute formulation at a concentration that is equal or greater than 15 mg/mL, optionally equal to or greater than 25 mg/mL. As a result, the formulations, solutions and pharmaceutical compositions described herein may be suitable for various modes of administration, including intranasal or oral routes of administration.

### DETAILED DESCRIPTION

When ranges are used herein, all combinations and sub-combinations of ranges and specific embodiments therein are intended to be included. The term “about” when referring to a number or a numerical range means that the number or numerical range referred to is an approximation within experimental variability (or within statistical experimental error), and thus the number or numerical range may vary. Typical experimental variabilities may stem from, for example, changes and adjustments necessary during scale-up from laboratory experimental and manufacturing settings to large scale, GMP manufacturing conditions as is known to those familiar with the art of pharmaceutical development and manufacturing. Such changes can vary between 1% and 10% of the stated number or numerical range.

The term “prodrug” refers to a molecule that may or may not have pharmacological activity on its own, but is chemically altered within the subject's body after administration either due to metabolism or due to exposure to a physiological medium or from biochemical processes in the cell, or otherwise to produce the pharmacologically active drug after administration. In the context of this disclosure, the pharmacologically active drug is a molecule that is able to inhibit, block or stall the replication of viral genome. Phosphoramidate, phosphonamidate, phosphoester or sugar-ester nucleotide or nucleoside pro-drugs such as remdesivir are all nucleotide or nucleotide pro-drugs. This is because the pharmacologically active drug is the nucleoside triphosphate. The term “pro-drug” referred to herein refers to a nucleoside or nucleotide pro-drug, i.e., a phosphoramidate, phosphonamidate, phosphoester or sugar-ester nucleotide or nucleoside pro-drugs.

The term “comprising” (and related terms such as “comprise” or “comprises” or “having” or “including”) has an open meaning and therefore a pharmaceutical composition comprising described features may comprise additional components in addition to the described features. The term “comprising” (and related terms) also may include those embodiments, for example, an embodiment of any formulation, solution, composition or method or the like., that “consist of” or “consist essentially of” the described features, i.e., are limited to the described features, where context allows.

Abbreviations used herein have their conventional meaning within the chemical and biological arts, unless otherwise indicated.

The term “oral formulation” is a finished dosage form and composition thereof, which is administered by mouth by an act of ingestion Oral formulation is taken to exclude intravenously administered formulations, or any dosage form and composition that can be injected, or inhaled or administered by other routes of administration, such as, rectal, topical or transdermal.

The term “intranasal formulation” is a finished dosage form and composition thereof, which is inhaled and/or administered by the nasal passage. Intranasal formulation is taken to exclude intravenously administered formulations or any dosage form and composition that can be taken orally or injected, or administered by other routes of administration, such as, rectal, topical or transdermal.

The term “injectable solution” is a finished dosage form and composition thereof which is administered by injection. Injectable solution is taken to exclude formulations that are formulated for oral and intranasal administration. The term injectable solution may be otherwise be described as a dilute ready-to-administer injectable solution.

The term “HLB value” is the hydrophilic lipophilic balance of a surfactant. The HLB value is a measure of how hydrophilic or lipophilic a surfactant is, with HLB numbers >10 have an affinity for water (hydrophilic) and number <10 have an affinity of oil (lipophilic). For non-ionic surfactants the HLB value is determined by the Griffin's method wherein HLB=20*Mₕ/M where Mₕ is the mass of the hydrophilic components, and M is the mass of the whole molecule. HLB values for ionic surfactants (i.e. wherein HLB values >20) can be determined using the Davies method.

The term “effective amount” or “therapeutically effective amount” refers to the amount of pro-drug (e.g. remdesivir) or metabolites thereof described herein that have adequate antiviral activity needed to bring about an acceptable outcome of the therapy as determined by the lessening of severity of the disease and/or complete remission of the disease as measurable by clinical, biochemical or other indicators that are familiar to those trained in the art. The therapeutically effective amount may vary depending upon the intended application (in vitro or in vivo), or the subject and the viral disease being treated, as well as, the disease condition being treated, e.g., the weight and age of the subject, the severity of the disease condition, the manner of administration and the like, which can readily be determined by one of ordinary skill in the art. The term also applies to a dose that will induce a particular response in target cells.

The specific dose will vary depending on the dosing regimen to be followed, whether it is administered in combination with other compounds, route and timing of administration, the tissue to which it is administered, and the physical delivery system in which it is carried.

The terms “treatment” and “treating” refer to an approach for obtaining beneficial or desired results including, but not limited to, therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. For prophylactic benefit, the compositions may be administered to a patient at risk of developing a particular disease, or to a patient reporting one or more of the physiological symptoms of a disease, even though a diagnosis of this disease may not have been made.

The term “therapeutic effect,” as that term is used herein encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

The term “subject” or “patient” refers to an animal, such as a mammal, for example, a human. The methods described herein can be useful in both human therapeutics and veterinary applications. In some embodiments, the patient is a mammal, and in preferred embodiments, the patient is human. For veterinary purposes, the term “subject” and “patient” include, but are not limited to, farm animals including cows, sheep, pigs, horses, and goats; companion animals such as dogs and cats; exotic and/or zoo animals; laboratory animals including mice, rats, rabbits, guinea pigs, and hamsters; and poultry such as chickens, turkeys, ducks, and geese.

Any method of treating a disease described herein, e.g., the method comprising administering to a subject in need thereof a therapeutically effective amount of the injectable solution or the pharmaceutical composition described herein may be rephrased or reformulated as “a medicament for use in the treatment of a disease”. In other words, if the disease is a certain type of viral infection the method of treatment can be rephrased to “a pharmaceutical composition or an injectable solution for use in the treatment of that type of viral infection”.

“wt. %” of a component described herein refers to the percentage weight of the component by weight of the ready-to-dilute formulation or injectable solution unless otherwise specified.

### Ready-to-Dilute Formulation

The present disclosure provides a ready-to-dilute formulation comprising
0.5-5 wt. % of a nucleoside or nucleotide pro-drug, wherein the nucleoside or nucleotide pro-drug is a phosphoramidate, phosphonamidate, phosphoester, or sugar-ester nucleoside or nucleotide pro-drug
2-15 wt. % pyrrolidone-containing compound; and
65-97.5 wt. % of one or more co-solvent(s).

In some examples, the nucleoside or nucleotide pro-drug is remdesivir and/or the pyrrolidone-containing compound is polyvinylpyrrolidone.

Ready-to-dilute formulations refer to formulations that are suitable for mixing with a diluent for administration. However, ready-to-dilute formulations described herein may otherwise simply be referred to as “formulations” or “concentrated formulations comprising the nucleoside or nucleotide pro-drug”. Typically, the concentrated formulation comprises the nucleoside or nucleotide pro-drug (e.g., remdesivir) at a concentration of at least 10 mg/mL, or at least 12.5 mg/mL, or at least 15 mg/mL. In preferred embodiments, the formulation is a solution, (i.e. a ready-to-dilute solution or a concentrated solution) of a nucleoside or nucleotide pro-drug

### Nucleoside or Nucleotide Pro-Drug, e.g., Remdesivir

The present formulations comprise a nucleoside or nucleotide pro-drug, wherein the nucleoside or nucleotide pro-drug is a phosphoramidate, phosphonamidate, phosphoester or sugar-ester nucleoside or nucleotide pro-drug. In some embodiments, the nucleotide or nucleoside may be selected from a modified adenosine, a modified guanidine, a modified cytidine, a modified uridine, or a modified deoxyuridine, a modified thymidine, a modified deoxyadenosine, a modified deoxyguanidine, a modified deoxycytidine. In some embodiments, the nucleotide or nucleoside may be selected from a modified adenosine, a modified guanidine, a modified cytidine, a modified uridine, in some examples, a modified adenosine. In some examples, wherein the modified adenosine is

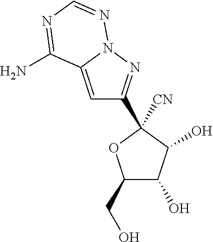 [SMILES: N#C[C@@]1(c2cc3c(N)ncnn3n2)O[C@H](CO)[C@@H](O)[C@H]1O]

In some embodiments, the phosphoramidate, phosphonamidate phosphoester or sugar ester is attached at the 5-OH, 3-OH, or 2-OH position on the ribose of the nucleoside or nucleotide, or the 5-OH or 3-OH position on the deoxyribose of the nucleoside or nucleotide. In some examples, a phosphoramidate is attached at the 5-OH position on the ribose of the nucleoside or nucleotide (e.g. remdesivir).

In preferred embodiments, the nucleotide or nucleoside prodrug is remdesivir.

Remdesivir is an adenosine phosphoramidate nucleoside anti-viral pro-drug otherwise known as GS-5734. Remdesivir has the following structure:

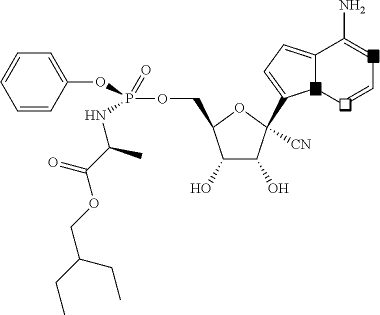 [SMILES: CCC(CC)COC(=O)[C@H](C)N[P@](=O)(OC[C@H]1O[C@@](C#N)(C2=CC=C3C(N)=CC=CC32)[C@H](O)[C@@H]1O)Oc1ccccc1]

Remdesivir and other anti-viral nucleotide or nucleoside phosphoramidates, phosphonamidates, phosphoesters and sugar-esters are described as a “pro-drug” since they need to be metabolized and converted to the mononucleotide triphosphate in the cell for their antiviral activity.

The ready-to-dilute formulation comprises 0.5-5 wt. % nucleoside or nucleotide pro-drug, (i.e. wherein the nucleoside or nucleotide pro-drug is a phosphoramidate, phosphonamidate, phosphoester or sugar-ester nucleoside or nucleotide pro-drug (e.g. remdesivir)). In some embodiments, the ready-to-dilute formulation comprises 0.75 to 4 wt. % nucleoside or nucleotide pro-drug (e.g. remdesivir), or 1 to 3 wt. %, or 1 to 2.5 wt. %, or 1.25 to 2.5 wt. % nucleoside nucleotide pro-drug (e.g. remdesivir). In some embodiments, the ready-to-dilute solution comprises at least 0.5 wt. % nucleoside or nucleotide pro-drug (e.g. remdesivir), or at least 0.75 wt. %, or at least 1 wt. %, or at least 1.1 wt. %, or at least 1.2 wt. %, or at least 1.25 wt. %, or at least 1.3 wt. %, or at least 1.4 wt. %, or at least 1.5 wt. %, or at least 1.6 wt. %, or at least 1.7 wt. %, or at least 1.8 wt. %, or at least 1.9 wt. %, or at least 2 wt. %, or at least 2.1 wt. %, or at least 2.2 wt. %, or at least 2.3 wt. % nucleoside or nucleotide pro-drug (e.g.

remdesivir). In some embodiments, the ready-to-dilute solution comprises less than 5 wt. % nucleoside or nucleotide pro-drug (e.g. remdesivir), or less than 4 wt. %, or less than 3 wt. %, or less than 2.75 wt. %, or less than 2.5 wt. %, or less than 2.4 wt. %, or less than 2.3 wt. %, or less than 2.2 wt. %, or less than 2.1 wt. %, or less than 2 wt. %, or less than 1.9 wt. %, or less than 1.8 wt. %, or less than 1.7 wt. %, or less than 1.6 wt. %, or less than 1.5 wt. %, or less than 1.4 wt. %, or less than 1.3 wt. % nucleoside or nucleotide pro-drug (e.g. remdesivir).

In some embodiments, the ready-to-dilute formulation comprises the nucleotide or nucleoside pro-drug (e.g. remdesivir) at a concentration of at least 12.5 mg/ml, or at least 13 mg/ml, or at least 14 mg/ml, or at least 15 mg/ml, or at least 17.5 mg/ml, or at least 20 mg/ml, or at least 22.5 mg/ml, or at least 25 mg/ml. High concentrations of anti-viral pro-drugs (e.g. remdesivir) are possible due to the effective solubilization of these compounds. This allows for low-dosage volumes of the ready-to-dilute formulation. In addition, due to the high dosage loading of the pro-drug (e.g. remdesivir), the ready-to-dilute formulation can also be used in a pharmaceutical composition which is suitable for intranasal or oral administration.

### Pyrrolidone Compound

The ready-to-dilute solution comprises 2-15 wt. % pyrrolidone-containing compound. The pyrrolidone-compound described herein may comprise any compound that contains at least one pyrrolidone group (i.e., a group otherwise known as 2-pyrrolidone or butyrolactam). The pyrrolidone-compound described herein may consist of a single pyrrolidone-compound or multiple pyrrolidone-compounds.

Due to the presence of at least the pyrrolidone-containing compound, the ready-to-dilute formulation comprises one or more Hydrogen-bond (H-bond) acceptors. It is believed that the pyrrolidone-compound is effective at stabilizing the pro-drug due to the presence of a H-bond acceptor. As a result, in an alternative version of this aspect, the pyrrolidone-compound may be replaced with a different H-bond acceptor.

In some embodiments, the ready-to-dilute solution comprises 3-13 wt. % pyrrolidone-containing compound, or 4-12 wt. % pyrrolidone-containing compound, or 4-11 wt. % pyrrolidone-containing compound, or 5-11 wt. % pyrrolidone-containing compound. In some embodiments, the ready-to-dilute solution comprises at least 2 wt. % pyrrolidone-containing compound, or at least 3 wt. %, or at least 4 wt. %, or at least 5 wt. %, or at least 6 wt. %, or at least 7 wt. %, or at least 8 wt. %, or at least 9 wt. %, or at least 10 wt. % pyrrolidone-containing compound. In some embodiments, the ready-to-dilute solution comprises less than 15 wt. % pyrrolidone-containing compound, or less than 14 wt. %, or less than 13 wt. %, or less than 12 wt. %, or less than 11 wt. %, or less than 10 wt. %, or less than 9 wt. %, or less than 8 wt. % or less than 7 wt. %, or less than 6 wt. % pyrrolidone-containing compound.

As demonstrated in the application examples, pyrrolidone compounds in combination with one or more cosolvent(s) can very effectively solubilize remdesivir (an example of a phosphoramidate or phosphonamidate nucleotide pro-drug). Furthermore, the presence of a pyrrolidone-compound contributes to the excellent chemical stability of the ready-to-dilute formulations described herein. The same excellent chemical stability is not observed for other non-aqueous formulations, where remdesivir is prone to hydrolysis.

In preferred embodiments, the pyrrolidone-containing compound is a pyrrolidone-containing polymer. The pyrrolidone-containing polymer comprises a plurality of pyrrolidone groups and is formed from polymerisation of a monomer comprising a pyrrolidone group.

In preferred embodiments, the pyrrolidone-containing compound and pyrrolidone-containing polymer comprises or consists of polyvinylpyrrolidone. Polyvinylpyrrolidone is a polymer formed from the monomer N-vinyl pyrrolidone. It is otherwise known as PVP, povidone or crospovidone in the art.

In some embodiments, the pyrrolidone-containing polymer (e.g., polyvinylpyrrolidone) has a weight average molecular weight between 2000 and 70000 g/mol. In some embodiments, the pyrrolidone-containing polymer (for example, polyvinylpyrrolidone) has a weight average molecular weight between 2000 and 60000 g/mol, or between 2000 and 54000 g/mol, or between 2000 and 34000 g/mol, or between 2000 and 25000 g/mol, or between 2000 and 11000 g/mol, or between 2000 and 6000 g/mol, or between 2000 and 3000 g/mol. The weight average molecular weight may be as determined using light scattering, for example, using ASTM D4001-13. In some embodiments, the pyrrolidone-containing polymer (e.g., polyvinylpyrrolidone) has a weight average molecular weight of less than 60000 g/mol, or less than 54000 g/mol, or less than 34000 g/mol, or less than 25000 g/mol, or less than 11000 g/mol, or less than 6000 g/mol. Lower weight pyrrolidone-containing polymers (for example, polyvinylpyrrolidone with a molecular weight of less than 6000 g/mol) are preferred in some examples since they show the best results in terms of solubility and/or stability.

In some embodiments, the polyvinylpyrrolidone has any suitable K-value. The K-value is a measure of the viscosity and is closely related to the intrinsic viscosity. K-value is obtained by dilute solution viscometry and by solving the Fikentscher equation (e.g. in accordance with DIN EN ISO 1628-1). In some embodiments, the polyvinylpyrrolidone has a K-value in the range of 10-100, more preferably in the range of 10-30, more preferably in the range of 10-20. In some embodiments, the polyvinylpyrrolidone has a K-value of 12 (K-12), 17 (K-17), 25 (K-25) or 30 (K-30). In some examples, the polyvinylpyrrolidone is K-12.

In alternative embodiments, or in addition to the pyrrolidone-containing compound comprising a pyrrolidone-containing polymer. the pyrrolidone-containing compound comprises an N-substituted C₁-C₆ alkyl pyrrolidone. In some embodiments, the N-substituted alkyl pyrrolidone is N-methyl pyrrolidone (e.g. otherwise known as NMP). As demonstrated in the examples described herein, N-methyl pyrrolidone is also shown to have an excellent solubilizing effect on remdesivir (i.e., an example of a phosphoramidate or phosphonamidate nucleotide pro-drug).

### Co-Solvent(s)

The ready-to-dilute formulation comprises one or more cosolvent(s), a term meant to include water miscible nonaqueous solvents. Such cosolvents preferably also have at least one H-bond acceptor group that can work alongside the pyrrolidone compound(s). The co-solvents can be used to improve the solubility and/or stability of the nucleoside or nucleotide pro-drugs (i.e., phosphoramidate or phosphonamidate or phosphoester or sugar-ester pro-drugs (e.g., remdesivir)) in solution, interact with the pyrrolidone compound to improve the chemical stability of the pro-drug (e.g., remdesivir) in solution and/or be used to ensure that the final injectable solution (i.e. once diluted) is stable, affords the required solubility of the nucleoside or nucleotide prodrug, and that the final diluted product is isotonic with blood.

In some embodiments, the ready-to-dilute formulation comprises 65 to 97.5 wt. % of one or more cosolvent(s). In some embodiments, the ready-to-dilute formulation comprises 70 to 95 wt. %, or 75 to 95 wt. %, or 80 wt. % to 90 wt. % of one or more cosolvent(s). In some embodiments, the ready-to-dilute formulation comprises at least 65 wt. % of one or more cosolvent(s), or at least 70 wt. %, or at least 75 wt. %, or at least 80 wt. %, or at least 85 wt. % one or more cosolvent(s). In some embodiments, the ready-to-dilute formulation comprises less than 97.5 wt. %, or less than 95 wt. %, or less than 92.5 wt. %, or less than 90 wt. %, or less than 87.5 wt. %, or less than 85 wt. % one or more cosolvent(s). In preferred embodiments, the one or more co-solvent(s) comprise PEG, a C2-C7 alcohol compound or a combination thereof.

In preferred embodiments, the one or more co-solvent comprises one or more PEG co-solvent(s). PEG as described herein refers to polyethylene glycol, also known as polyethylene oxide (PEO) or polyoxyethylene (POE), and has a structure that can be commonly expressed as H—(O—CH₂—CH₂)ₙ—OH, where the subscript ‘n’ is an integer to represent the number of repeating ‘oxyethylene’ groups. The ether bonds and the terminal hydroxyl (OH) group can both act as H-bond acceptors. In the examples demonstrated herein, formulations comprising PEG and a pyrrolidone compound (e.g. PVP) were found to have excellent chemical stability. Example formulations comprising PEG and a pyrrolidone compound (e.g. PVP) were also shown to effectively solubilize remdesivir.

In some embodiments, the formulation comprises one or more PEG co-solvent(s) in an amount of 50 to 97.5 wt. %. In some embodiments, the ready-to-dilute formulation comprises one or more PEG cosolvent(s) in an amount of 60 wt. % to 90 wt. %, or from 65 wt. % to 85 wt. %, or from 70 wt. % to 80 wt. %. In some embodiments, the formulation comprises one or more PEG cosolvent(s) in an amount of at least 50 wt. %, or at least 55 wt. %, or at least 60 wt. %, or at least 65 wt. %, or at least 70 wt. %, or at least 75 wt. %. In some embodiments, the formulation comprises one or more PEG cosolvent(s) in an amount that is less than 97.5 wt. %, or less than 95 wt. %, or less than 90 wt. %, or less than 87.5 wt. %, or less than 85 wt. %, or less than 82.5 wt. %, or less than 80 wt. %, or less than 77.5 wt. %, or less than 75 wt. %. As shown herein, example formulations comprising PEG co-solvents and pyrrolidone compound (PVP) had excellent chemical stability and effectively solubilized remdesivir, (i.e., nucleotide pro-drug).

In some embodiments, the one or more PEG co-solvent(s) have a number average molecular weight from 200 to 600. In some embodiments, the one or more co-solvent(s) comprise PEG 300 and/or PEG 400. PEG 400 has a number average molecular weight of between 380 to 420 g/mol, and PEG 300 has a number average molecular weight of 285 g/mol to 315 g/mol.

In some embodiments, the ready-to-dilute solution comprises PEG 300 and/or PEG 400. In some embodiments the ready-to-dilute solution comprises PEG 300 and PEG 400 in a ratio of 0.5:1 to 2.5:1. In some embodiments, the ready-to-dilute formulation comprises 20 to 50 wt. % PEG 300 and/or 20 to 50 wt. % PEG 400. In some embodiments, the ready to-dilute formulation comprises 25 wt. % to 45 wt. % PEG 300 and/or 20 wt. % to 50 wt. % PEG 400. In some embodiments, the ready-to-dilute formulation comprises at least 20 wt. % PEG 300, or at least 25 wt. %, or at least 30 wt. %, or at least 35 wt. %, or at least 40 wt. %, or at least 45 wt. % PEG 300. In some embodiments, the ready-to-dilute formulation comprises less than 50 wt. % PEG 300, or less than 47.5 wt. %, or less than 45 wt. %, or less than 42.5 wt. %, or less than 40 wt. %, or less than 37.5 wt. %, or less than 35 wt. %, or less than 32.5 wt. %, or less than 30 wt. %, or less than 27.5 wt. %, or less than 25 wt. %, or less than 22.5 wt. % PEG 300. In some embodiments, the ready-to-dilute formulation comprises at least 20 wt. % PEG 400, or at least 25 wt. %, or at least 30 wt. %, or at least 35 wt. %, or at least 40 wt. %, or at least 45 wt. % PEG 400. In some embodiments, the ready-to-dilute formulation comprises less than 50 wt. % PEG 400, or less than 47.5 wt. %, or less than 45 wt. %, or less than 42.5 wt. %, or less than 40 wt. %, or less than 37.5 wt. %, or less than 35 wt. %, or less than 32.5 wt. %, or less than 30 wt. %, or less than 27.5 wt. %, or less than 25 wt. %, or less than 22.5 wt. % PEG 400.

In some embodiments, the one or more co-solvent(s) comprises one or more C2-C7 alcohol compound. In some embodiments, the one or more C2-C7 alcohol compound may be selected from ethanol, benzyl alcohol, tertiary butyl alcohol (or tert-butyl alcohol) or propylene glycol. In some embodiments, the one or more C2-C7 alcohol is selected from ethanol, benzyl alcohol or propylene glycol. In some embodiments, the ready-to-dilute formulation comprises one or more C2-C7 alcohol(s) in an amount up to about 25 wt. %. In some embodiments, the ready-to-dilute formulation comprises one or more C2-C7 alcohol in an amount between 5 wt. % and 22 wt. %, or between 5 wt. % and 15 wt. %, or between 5 wt. % and 10 wt. %. In some embodiments, the ready-to-dilute formulation comprises one or more

C2-C7 alcohol(s) in an amount of at least 1 wt. %, or at least 2 wt. %, or at least 3 wt. %, or at least 4 wt. %, or at least 5 wt. %, or at least 6 wt. %, or at least 7 wt. %, or at least 8 wt. %, or at least 9 wt. %, or at least 10 wt. %, or at least 11 wt. %, or at least 12 wt. %, or at least 13 wt. %, or at least 14 wt. %, or at least 15 wt. %, or at least 16 wt. %, or at least 17 wt. %, or at least 18 wt. %, or at least 19 wt. %, or at least 20 wt. %. In some embodiments, the ready-to-dilute formulation comprises one or more C2-C7 alcohol in an amount of less than 25 wt. %, or less than 22.5 wt. %, or less than 20 wt. %, or less than 17.5 wt. %, or less than 15 wt. %, or less than 12.5 wt. %, or less than 10 wt. %, or less than 9 wt. %.

In some embodiments, the one or more co-solvent comprises propylene glycol. In some embodiments, the one or more co-solvent(s) comprises propylene glycol in an amount up to about 25 wt. %. In some embodiments, the ready-to-dilute formulation comprises propylene glycol in an amount between 5 wt. % and 22 wt. %, or between 5 wt. % and 15 wt. %, or between 5 wt. % and 10 wt. %. In some embodiments, the ready-to-dilute formulation comprises propylene glycol in an amount of at least 1 wt. %, or at least 2 wt. %, or at least 3 wt. %, or at least 4 wt. %, or at least 5 wt. %, or at least 6 wt. %, or at least 7 wt. %, or at least 8 wt. %, or at least 9 wt. %, or at least 10 wt. %, or at least 11 wt. %, or at least 12 wt. %, or at least 13 wt. %, or at least 14 wt. %, or at least 15 wt. %, or at least 16 wt. %, or at least 17 wt. %, or at least 18 wt. %, or at least 19 wt. %, or at least 20 wt. %. In some embodiments, the ready-to-dilute formulation comprises propylene glycol in an amount of less than 25 wt. %, or less than 22.5 wt. %, or less than 20 wt. %, or less than 17.5 wt. %, or less than 15 wt. %, or less than 12.5 wt. %, or less than 10 wt. %, or less than 9 wt. %. The presence of propylene glycol can be used to further ensure that there is sufficient solubility of the nucleoside or nucleotide pro-drug (i.e., the phosphoramidate, phosphonamidate or polyester or sugar-ester prodrug) and that there is no precipitation following dilution and that the resultant final, dilute, ready-to-administer injectable solution has an osmolality that is isotonic with blood.

In some embodiments, the one or more co-solvent(s) comprises one or more PEG cosolvent(s) in combination with one or more C2-C7 alcohol compound (i.e. in the amounts as described herein). In some embodiments, the one or more co-solvent(s) comprises PEG 300, PEG 400 and one or more C2-C7 alcohol compound. In some embodiments, the one or more co-solvent(s) comprises PEG 300, PEG 400, and propylene glycol (i.e., in the amounts described herein). In some embodiments, the one or more co-solvent(s) consist of PEG 300, PEG 400, and propylene glycol.

In preferred embodiments, the ready-to-dilute formulation is non-aqueous or substantially non-aqueous. Substantially non-aqueous may mean that the ready-to-dilute formulation comprises less than 0.5 wt. % water, or less than 0.1 wt. % water.

### Surfactant(s)

In some embodiments, the ready-to-dilute formulation comprises one or more surfactant(s). The one or more surfactant(s) can further improve the solubility of the nucleoside or nucleotide pro-drug (i.e., the phosphoramidate or phosphonamidate or phosphoester or ester pro-drug (e.g., remdesivir)) in the injectable solution, when diluted with an aqueous diluent.

In some embodiments, the one or more surfactant(s) has a HLB value from 10-20, optionally from 12 to 18, or optionally from 14 to 16.

In some embodiments, the one or more surfactant(s) is or comprises: a polysorbate, a polyoxyl castor oil, cremophor, polyoxyethylene (20) sorbitan monooleate, polyethylene glycol sorbitan monooleate, polyoxyethylenesorbitan monooleate, or a block copolymer of poly(ethylene oxide) (PEO) and poly(propylene oxide) (PPO), such as, poloxamer, or a combination thereof. In some embodiments, the one or more surfactant(s) is a polysorbate or a polyoxyl castol oil, for example, polyoxyl 35 castol oil. In some embodiments, the one or more surfactant(s) is polysorbate, for example, polysorbate 20, 40, 60 or 80 (otherwise known as Tween 20, 40, 60 or 80). In some embodiments and examples, the polysorbate is polysorbate 80.

In some embodiments, the ready-to-dilute formulation comprises the one or more surfactant(s) in an amount up to 15 wt. %. In some embodiments, the ready-to-dilute formulation comprises one or more surfactant(s) in an amount from 2 wt. % to 13 wt. %, or from 4 wt. % to 12 wt. %, or from 5 wt. % to 11 wt. %, or from 6 wt. % to 10 wt. %, or from 7 wt. % to 9 wt. %. In some embodiments, the ready-to-dilute formulation comprises one or more surfactant(s) in an amount less than 15 wt. %, or less than 14 wt. %, or less than 13 wt. %, or less than 12 wt. %, or less than 11 wt. %, or less than 10 wt. %, or less than 9 wt. %, or less than 8 wt. %, or less than 7 wt. %, or less than 6 wt. %, or less than 5 wt. %, or less than 4 wt. %, or less than 3 wt. %, or less than 2 wt. %, or less than 1 wt. %. In some embodiments, the ready-to-dilute formulation comprises one or more surfactant(s) in an amount greater than 1 wt. %, or greater than 2 wt. %, or greater than 3 wt. %, or greater than 4 wt. %, or greater than 5 wt. %, or greater than 6 wt. %, or greater than 7 wt. %, or greater than 8 wt. %.

### Absence of Cyclodextrin

In preferred embodiments, the ready-to-dilute formulation is free from any cyclodextrin compound. In some embodiments, the ready-to-dilute formulation is free from alpha, beta, and/or gamma cyclodextrin compounds. In some embodiments, the ready-to-dilute formulaton is free from any derivatized and/or modified cyclodextrin compounds, such as, hydroxylpropyl B-cyclodextrin and sulfobutylether 8-cyclodextrin and/or their salts. This is advantageous because cyclodextrins can accumulate in the kidney at high doses.

Free from pH Modifiers or Buffers

In preferred embodiments, the ready-to-dilute formulation is free from any pH modifiers or buffers and/or salts. pH modifiers, adjusters or buffers as described herein may include hydrochloric acid, sodium hydroxide, tris (tromethamine) acetate, citrate, tartaric acid or salts thereof, lactic acid and salts thereof, phosphates, benzoates, bicarbonate, carbonates, sulphates, sodium chloride, potassium chloride, calcium chloride, tromethamine or a combination thereof. Absence of salts in the formulation is particularly advantageous to maintain hemodynamic stability for hospitalized and critically ill patients receiving remdesivir who might also be receiving other intravenous (IV) injections and infusions containing such buffer salts. The absence of salts may be beneficial for eliminating the risk of salt overload which may cause hemodynamic instability, which may be especially necessary for patients that are already receiving other IV drugs and infusion fluids containing buffers and/or salts.

### Stability

The ready-to-dilute formulations described herein have good shelf-life and/or stability. The stability of the pharmaceutical composition of the present invention may be monitored any suitable method, and in some examples, by HPLC. The stability may be determined by establishing the initial amount of nucleoside or nucleotide pro-drug (i.e., phosphoramidate or phosphonamidate or phosphoester or sugar-ester pro-drug (e.g. remdesivir)) (i.e. at time=0), and then measuring the amount of nucleoside or nucleotide pro-drug (e.g. remdesivir) remaining after a certain time thereafter and comparing the two values to determine the % of non-degraded compound. The initial amount of nucleoside or nucleotide pro-drug (e.g. remdesivir) may correspond to the amount present immediately after mixing all the components of the ready-to-dilute solution. In some embodiments, the method of tracking stability can constitute comparing the purity of nucleoside or nucleotide pro-drug (e.g. remdesivir) in the ready-to-dilute formulation against that of a freshly prepared standard solution to calculate the % of non-degraded nucleoside or nucleotide pro-drug (e.g. remdesivir) in the product for any given sample. Samples that are stored and analyzed over various periods of time can provide a quantitative profile of the purity of the nucleoside or nucleotide pro-drug (e.g. remdesivir) over time.

The ready-to-dilute formulations described herein have good shelf-life and/or stability at ambient temperatures and also under accelerated (stressed stability due to elevated temperature and/or humidity) conditions. In certain embodiments, at least 90% by weight of nucleoside or nucleotide pro-drug (e.g. remdesivir) is present (i.e., is non-degraded) in the concentrated, ready-to-dilute solution formulation after being stored for 30 days or for 2 months, or for 6 months, or for 9 months, or for 24 months, or for 36 months at 25° C., as compared to the initial amount of the compound of nucleoside or nucleotide pro-drug (e.g. remdesivir) in the same formulation. In certain embodiments, at least 92%, or at least 94%, or at least 96%, or at least 98%, or at least 99%, or at least 99.5% of nucleoside or nucleotide pro-drug (e.g. remdesivir) is present in the composition after being stored for 30 days, or for 2 months, or for 6 months, or for 9 months, or for 24 months, or for 36 months at 25° C., as compared to the initial amount of nucleoside or nucleotide pro-drug (e.g. remdesivir) in the same formulation.

The ready-to-dilute formulations described herein also have good shelf-life and/or stability at elevated temperatures. In certain embodiments, at least 90% by weight of nucleoside or nucleotide pro-drug (e.g. remdesivir) is present (i.e. is non-degraded) in the ready-to-dilute formulation after being stored for 14 days, or for 30 days, or for 2 months or for 6 months at 40° C., compared to the initial amount of the compound of nucleoside or nucleotide pro-drug (e.g. remdesivir) in the ready-to-dilute formulation. In certain embodiments, at least 92%, or at least 94%, or at least 96%, or at least 98%, or at least 99%, or at least 99.5% of nucleoside or nucleotide pro-drug (e.g. remdesivir) is present in the ready-to-dilute formulation after being stored for 14 days, or for 30 days, or for 2 months or for 6 months at 40° C., compared to the initial amount of the compound of nucleoside or nucleotide pro-drug (e.g. remdesivir) in the ready-to-dilute formulation.

The ready-to-dilute formulations described herein have good shelf-life and/or stability when subjected to steam-sterilization. In certain embodiments, at least 90% by weight of nucleoside or nucleotide pro-drug (e.g. remdesivir) is present (i.e. is non-degraded) in the ready-to-dilute formulation after being heated for 30 minutes at 121° C., as compared to the initial amount of the compound of pro-drug (e.g. remdesivir) in the ready-to-dilute formulation. In certain embodiments, at least 92%, or at least 94%, or at least 96%, or at least 98% by weight of pro-drug (e.g. remdesivir) is present (i.e. is non-degraded) in the ready-to-dilute formulation after being heated for 30 minutes at 121° C., as compared to the initial amount of the compound of nucleoside or nucleotide pro-drug (e.g. remdesivir) in the ready-to-dilute formulation.

Dosage Volume, Vial and/or Packaging

In some embodiments, the ready-to-dilute formulation is packaged in a vial, e.g., a glass vial. In some embodiments, the ready-to-dilute formulation is packaged into a type-I glass vial (e.g. with a stopper and flip-off crimp-cap). Prior to administration, the entire dose volume can be withdrawn using an aseptic needle and syringe assembly and injected into the infusion vial or bag and mixed. In some embodiments, the ready-to-dilute formulation is packaged in a container that is suitable for direct transfer to the diluent, e.g., without the need for first withdrawing the dose and then transferring it. In some embodiments, the ready-to-dilute formulation is filled into a vial that is connected directly to an aqueous diluent (e.g., an IV infusion fluid) by an adaptor, for example, of the type similar to Add-Vantage™ vials. In some embodiments, the glass vial has a specially constructed cap assembly that fits directly into a specially constructed port in a flexible package containing the diluent, which is another vial+bag system where direct transfer can be possible. In some other embodiments, the ready-to-dilute solution may be filled into one of the two juxtaposed chambers in a flexible container, such as, a two-chambered IV infusion bag, where the diluent is filled into the other. In such a 2-chambered bag, a temporary barrier separates the concentrated ready-to-dilute solution from the diluent. The temporary barrier can be easily removed for direct mixing of the two liquids by a simple maneuver without exposing the contents of the packaging to the outside. In some embodiments, the ready-to-dilute formulation is filled into a first bag or bottle, with the aqueous diluent (e.g., an IV infusion fluid) filled into a second bag or bottle, wherein the first bag or bottle and second bag or bottle are separated by a temporary barrier which can be removed such that the components are mixed prior to administration. This is particularly useful when the ready-to-dilute solution is stable in the flexible packaging and the active ingredient is administered at a fixed dose, without the need for dose titration for various patients.

In some embodiments, the dosage volume of the ready-to-dilute formulation is between 1 mL and 10 mL. In some embodiments the dosage volume of the ready-to-dilute formulation is between 2.5 mL and 9 mL, or between 5 mL and 8 mL. In some embodiments, the dosage volume of the ready-to-dilute formulation is less than 10 mL, or less than 9 mL, or less than 8 mL, or less than 7 mL, or less than 6 mL, or less than 5 mL, or less than 4 mL, or less than 3 mL. Low dosage volumes are advantageous for increased ease and flexibility when preparing an injectable solution. Low dosage volumes also have reduced shipping costs. Low dosage volumes are possible due to the high concentration of nucleoside or nucleotide pro-drug (e.g. remdesivir) in the ready-to-dilute formulations as a result of effective solubilization and increase in solubility.

### Injectable Solution

The present invention provides for an injectable solution comprising the ready-to-dilute formulation described herein (i.e. according to the first aspect or fourth aspect), and an aqueous diluent.

The injectable solution may be formulated for intravenous injection or intramuscular injection, preferably intravenous injection. In preferred embodiments, the aqueous diluent is an intravenous medium fluid. In some embodiments, the aqueous diluent comprises one or more tonicity adjusters. In some embodiments, the aqueous diluent comprises surfactants and/or solubilizing substances in addition to tonicity adjusters. This may help to further maintain sufficient solubility of the active even after dilution.

In some embodiments, the aqueous diluent comprises water, NaCl solution and/or dextrose solution. In some embodiments, the aqueous diluent is or consists of water (i.e. sterile water for injection), 0.9% NaCl (normal saline), 0.45% NaCl (half-normal saline), 0.225% NaCl (quarter normal saline) or 5% dextrose solution. In preferred embodiments, the aqueous diluent is or consists of water (i.e. sterile water for injection), 0.45% NaCl or 5% dextrose solution. These components can be mixed with the ready-to-dilute formulation to give an injectable solution that is isotonic with blood.

In some embodiments, the aqueous diluent further comprises a co-solvent and/or a surfactant as otherwise described herein. In some embodiments, the aqueous diluent comprises a co-solvent, e.g., PEG (e.g. PEG 400 and/or PEG 300), in an amount from 0.75 wt. % to 5 wt. %, or in an amount from 1 wt. % to 3 wt. %. In some embodiments, the aqueous diluent comprises a surfactant, e.g., polysorbate 80, in an amount from 0.1 wt. % to 1 wt. %, or in an amount from 0.2 wt. % to 0.5 wt. %.

In some embodiments, the pH of the injectable solution is between 4 and 8, more preferably between 5 and 7, and in some examples between 5 and 6. In some embodiments, the tonicity of the injectable solution is 300±50 mOsm and, more preferably, 300±25 mOsm

In some embodiments, the ratio of ready-to-dilute solution to aqueous diluent is 1:8 to 1:100, or from 1:8 to 1:50, or from 1:8 to 1:25, or from 1:8 to 1:18, or from 1:10 to 1:17, or from 1:12 to 1:16, or from 1:13 to 1:15.

In some embodiments, the dose-volume of the injectable solution (e.g., a dilute, ready-to-administer injectable solution) is from 75 mL to 300 mL, or from 100 mL to 250 mL. In some examples, the dose-volume of the injectable solution is 100 mL or 250 mL. In some embodiments the amount of compound of nucleoside or nucleotide pro-drug (i.e., phosphoramidate, phosphoramidite, phosphoester or sugar-ester nucleoside or nucleotide pro-drug (e.g. remdesivir)) in the dosage form of the injectable solution is from 20 to 300 mg, or from 50 to 250 mg, or from 100 mg to 200 mg of nucleoside or nucleotide pro-drug. In some examples, the nucleoside or nucleotide prodrug is remdesivir.

In some embodiments, the injectable solution comprises 1-10% w/w of the ready-to-dilute formulation and 90-99% w/w of the aqueous diluent. In some embodiments, the injectable solution comprises from 5-10% w/w of the ready-to-dilute formulation and 90-95% w/w of the aqueous diluent (e.g. water). For example, a 100 mL injectable solution may comprise between 90-95 mL water (e.g. 93.3 mL water) and between 5-10 mL of ready-to-dilute formulation (e.g. 6.7 mL). In other embodiments, the injectable solution comprises from 2-4% w/w of the ready-to-dilute formulation and 96-98% w/w of the aqueous diluent (e.g. half-saline or 5% dextrose solution). For example, a 250 mL injectable solution may comprise between 240-245 mL water, half-saline or 5% dextrose solution, and between 5-10 mL of ready-to-dilute formulation.

In some embodiments, the injectable solution may comprise between 0.025 wt. % to 2 wt. % nucleoside or nucleotide pro-drug (i.e., wherein the nucleoside or nucleotide pro-drug is a phosphoramidate, phosphonamidate, phosphoester or sugar-ester nucleoside or nucleotide pro-drug, e.g. remdesivir), or from 0.3 wt. % to about 1.5 wt. % nucleoside or nucleotide pro-drug (e.g. remdesivir). In some examples, the injectable solution may comprise between 0.075 wt. % to about 1.125 wt. % nucleoside or nucleotide pro-drug (e.g. remdesivir).

In some embodiments, the concentration of nucleoside or nucleotide pro-drug (e.g. remdesivir) in the injectable solution is from 0.25 mg/mL to 2 mg/mL, more preferably from 0.4 mg/mL to 1 mg/mL.

In some embodiments, the injectable solution may comprise 0.1 wt. % to 1 wt. % polyvinylpyrrolidone, or in some examples, from 0.2 wt. % to 0.7 wt. % polyvinylpyrrolidone.

In some embodiments, the injectable solution may comprise from 3 wt. % to 8 wt. % of one or more PEG cosolvent(s), or from 4 wt. % to 6.5 wt. %, or from 5 wt. % to 6 wt. % of one or more PEG cosolvent(s). In some embodiments, the injectable solution may comprise 2 wt. % to 3 wt. % PEG 300 and 2 wt. % to 3 wt. % PEG 400.

In some embodiments, the injectable solution may comprise one or more surfactants, e.g. a polysorbate and more specifically polysorbate 80, in an amount up to 1 wt. %, or from 0.2 wt. % to 1 wt. %, or from 0.4 wt. % to 0.8 wt. %.

In some embodiments, the injectable solution may comprise one or more C2 to C7 alcohols, e.g. propylene glycol, in an amount up to 1 wt. %, or from 0.2 wt. % to 1 wt. %, or from 0.4 wt. % to 0.8 wt. %.

In some embodiments, the injectable solution may comprise the aqueous diluent in an amount from 90 wt. % to 99.5%, or from 90 wt. % to 99.9 wt. %, or from 90 wt. % up to 100 wt. %.

### Solution

Also described herein is a solution (i.e., a concentrated solution) comprising a nucleoside or nucleoside or nucleotide pro-drug, polyvinylpyrrolidone and PEG, wherein the nucleoside or nucleotide pro-drug is a phosphoramidate, phosphonamidate, phosphoester or sugar-ester nucleoside or nucleotide pro-drug (e.g. remdesivir). The solution may comprise the nucleoside or nucleotide pro-drug (e.g. remdesivir), polyvinylpyrrolidone and PEG in the same amounts and/or as is elsewhere described herein for the ready-to-dilute formulation. The solution may be suitable for administration as an oral solution. In some embodiments, the solution comprises remdesivir, polyvinylpyrrolidone and PEG, wherein the PEG is PEG 300 and/or PEG 400. In some embodiments, the solution comprises remdesivir, polyvinylpyrrolidone and PEG 300.

The solutions may be suitable for administration as an oral solution.

### Pharmaceutical Composition

Also described herein is a pharmaceutical composition comprising the ready-to-dilute formulation (i.e. according to the first aspect or fourth aspect) or solutions described herein (i.e. according to the fifth aspect), optionally in the presence of one or more pharmaceutically acceptable excipients.

The pharmaceutically acceptable excipients may be selected from chelating agents, amino acids, sugars, mucolytic agents, buffers, pH and tonicity adjusters, co-solvents (i.e., in addition to co-solvents present in the ready-to-dilute formulation), polymers (i.e. in addition to a pyrrolidone-containing polymer in the ready-to-dilute formulation, if present) and combinations thereof.

In some embodiments, the chelating agent may be selected from EDTA and salts thereof, citric acid, malic acid, malonic acid, oxalic acid, succinic acid, tartaric acid or a combination thereof.

In some embodiments, the amino acid may be selected from histidine, glutamic acid, lysine, arginine, or a combination thereof.

In some embodiments, the sugar may be selected from dextrose, sucrose, mannose, mannitol or a combination thereof.

In some embodiments, the mucolytic agent may be N-acetyl-cysteine. Mucolytic agents may aid delivery of the pharmaceutical formulation to the site of infection in the lungs, by improving permeability and access to the infection across mucous barriers.

In some embodiments, the buffer may be selected from hydrochloric acid, sodium hydroxide, tris, acetate, citrate, tartrate, phosphate, benzoate, bicarbonate, sodium chloride, potassium chloride or a combination thereof.

The pH adjuster may include any suitable acid or base. In an embodiment, the pH adjuster is HCl or NaOH.

In some embodiments, the tonicity adjusters comprise one or more sugars. In some embodiments, the one or more sugars are selected from glucose or dextrose. In some embodiments, the tonicity adjusters comprise one or more salts. In some embodiments, the one or more salts are selected from NaCl or KCI. In some embodiments, the tonicity adjuster is selected from NaCl, KCI, mannitol, glycerin, glucose or dextrose.

In some embodiments, the co-solvent (i.e., in addition to the co-solvents present in the ready-to-dilute formulation) may comprise one, two, three, or four or more co-solvents. The co-solvent may be selected from polyethylene glycol (PEG) (e.g. PEG 400), glycerol, DMSO, ethanol, propylene glycol, polypropylene glycol, benzyl alcohol, cetostearyl alcohol, benzylbenzoate, corn syrup, acacia syrup, glucose syrup, acetyltributyl citrate, lactic acid, acetic acid, ethylacetate, benzoic acid, water; mineral oils; hydrogenated edible oils and non-hydrogenated edible oils, such as, anise oil, cinnamon oil, coriander oil, castor oil, clove oil, coconut oil, corn oil, cottonseed oil, lemon oil, soybean oil, sesame oil, vegetable oil, or a combination thereof.

In some embodiments, the polymer comprises one, two, three, or four or more polymers. In some embodiments, the polymer is selected from methyl acrylate-methacrylic acid copolymers, cellulose acetate phthalate, cellulose acetate succinate, hydroxypropyl methyl cellulose phthalate, hydroxypropyl methyl cellulose acetate succinate, polyvinyl acetate phthalate, shellac, cellulose acetate trimetallate, sodium alginate, zein, polyvinylpyrrolidone, poly(caprolactone) (PCL), poly(lactic-co-glycolic acid) (PLGA), poly(lactic acid) (PLA), (polyhydroxybutyrate) (PHB), poly(methysilsesquioxane) (PMSQ) or combinations thereof. In some embodiments, the polymer is a biodegradable (e.g. hydrolyzable) polymer, for example, PCL, PLGA, PLA or PHB.

In some embodiments the pharmaceutical excipient comprises a wetting agent. In some embodiments, the wetting agent is a selected from benzalkonium chloride, poloxamers (e.g. poloxamer 188,

In some embodiments, the pharmaceutical composition consists of the ready-to-dilute formulation (e.g. the ready-to-dilute formulation can be administered directly to a patient) without the addition of further pharmaceutically acceptable excipients.

In some embodiments, the pharmaceutical composition is a liquid formulation. In some embodiments, the pharmaceutical composition is an oral formulation. In some embodiments the amount of liquid formulation is in a dosage form from about 1 mL to about 40mL of liquid formulation, for example about 3 mL to about 35 mL, or about 5 mL to about 30 mL, or about 10 mL to about 30 mL, or about 10 mL to about 25 mL, or about 15 mL to about 25 mL. The oral formulation may be formulated as an oral solution or a liquid-filled capsule comprising the liquid formulation, wherein the capsule may comprise a gelatin or hypomellose shell.

In some embodiments, the pharmaceutical composition comprises the nucleotide or nucleotide pro-drug (i.e., the phosphoramidate, phosphonamidate, phosphoester or sugar-ester nucleoside or nucleotide pro-drug (e.g. remdesivir)) at a concentration of at least 12.5 mg/ml, or at least 13 mg/ml, or at least 14 mg/ml, or at least 15 mg/ml, or at least 17.5 mg/ml, or at least 20 mg/ml, or at least 22.5 mg/ml, or at least 25 mg/ml.

In an embodiment the pharmaceutical composition further comprises sweeteners, common taste-masking agents, flavors and/or colors, the addition of which may make the composition more palatable. In some embodiments, the oral formulation may be formulated in the form of a medicine, a syrup, an elixir, syrup or a suspension.

### Method of Treatment

Disclosed herein, is a method of treating a viral infection, the method comprising administering to a subject in need thereof a therapeutically effective amount of the injectable solution described herein. In an embodiment, the injectable solution is administered intravenously or intramuscularly. In an embodiment, the injectable solution is administered intravenously.

Also disclosed herein is a method of treating a viral infection, the method comprising administering to a subject in need thereof a pharmaceutical composition comprising the ready-to-dilute formulation or solution described herein. In some embodiments, the pharmaceutical composition may be administered orally or intranasally.

In some embodiments, the viral infection described herein is an RNA viral infection. In some embodiments, virus causing the viral infection is a human-disease causing virus. In some embodiments, the virus may be coronavirus, respiratory syncytial virus, ebola, hepatitis, junin, lassa fever, adenovirus, astrovirus, dengue, orthomyxovirus, Hepatitis Virus (HV) type, disease-causing picornavirus, Ebola, SARS, MERS, alpha, papillomavirus, chikungunya,

Epstein-barr, respiratory syncytial virus and other pneumovirus, influenza, rhinovirus, mumps, polio measles and retrovirus including adult Human T-cell lymphotropic virus type 1 (HTLV-1) and human immunodeficiency virus (HIV).

In some embodiments, the RNA virus may be a coronavirus. In some embodiments, the coronavirus is a coronavirus causing disease in humans. In some embodiments, the virus may be a coronavirus that causes disease in non-human animal species, such as, the feline infectious peritonitis virus, the porcine deltacorona virus.

In some embodiments, the viral infection is a coronavirus infection. The coronavirus infection may be caused by any type or strain of coronavirus. In some embodiments, the coronavirus infection may be an alphacoronavirus infection or a betacoronavirus infection, preferably a betacoronavirus. The betacoronavirus may have an A lineage, a B lineage, a C lineage or a D lineage, for example, a B lineage. In preferred embodiments, the coronavirus infection may be COVID-19, otherwise known as SARS-COV-2 or 2019-nCoV.

In some embodiments, the injectable solution or pharmaceutical composition described herein may be administered in circumstances where there is an anticipated risk of infection for prophylactic use to prevent such infection or, at the very least, to prevent severe manifestations of the disease. In some embodiments, the injectable solution or pharmaceutical composition is administered after a positive test for SARS-COV-2, for example, a positive PCR test or a positive lateral flow antigen test.

In some embodiments, the injectable solution or pharmaceutical composition described herein may be administered after the onset of COVID-19 symptoms. The one or more symptoms may include cough, sore throat, a high temperature or fever, loss of smell or taste, difficulty in breathing, tiredness, muscle pain, chest pain, runny nose, headache, chills, or any combination thereof. The injectable solution or pharmaceutical composition may alleviate one or more of these COVID-19 symptoms.

In some embodiments, the viral infection is a hepatitis infection. The hepatitis infection may be hepatitis A, B, C, D or E. The hepatitis infection may be acute hepatitis, fulminant hepatitis or chronic hepatitis.

In some embodiments, the injectable solution or pharmaceutical composition described herein is administered from every 4 hours up to every 72 hours. In an embodiment, the pharmaceutical composition described herein is administered every 4 hours, or up to every 8 hours, or up to every 12 hours, or up to every 16 hours, or up to every 24 hours, or up to every 48 hours, or up to every 36 hours, or up to every 72 hours. The injectable solution or pharmaceutical composition may be administered more frequently if the symptoms are more severe. An effective amount of the injectable solution or pharmaceutical composition described herein may be administered in either single or multiple doses. The multiple doses may be taken at the same time, or at different timepoints in the day (e.g. once, twice, three times, four times, five times or even six times a day).

In preferred embodiments, the injectable solution is administered intravenously once daily. The injectable solution may be administered intravenously once daily for any number of days, for example, for 1 day, 2 days, 3 days, 4 days, 5 days, 6 days, 7 days, 8 days, 9 days, 10 days. In some embodiments, one dose of the injectable solution may be administered over a period of 20 minutes to 180 minutes, or over a period of 30 minutes to 120 minutes.

The amount of nucleoside or nucleotide pro-drug (i.e., phosphoramidate phosphonamidate, phosphoester or sugar-ester nucleoside or nucleotide pro-drug (e.g. remdesivir)) to be administered (i.e. the dosage) is dependent on the specific viral infection being treated, the mammal being treated, the severity of the disorder or condition, the rate of administration and its effective inhibitory concentration (IC₅₀) against the specific virus infection being treated. In an embodiment, the dosage of the nucleoside or nucleotide pro-drug (e.g. remdesivir) is from about 2 to 20 mg/kg or from 2.5 mg/kg to 10 mg/kg, or from 2.5 mg/kg to 5 mg/kg.

### Preferred Embodiments of the Invention

The following lettered embodiments are preferred embodiments of the present invention.
A. A ready-to-dilute formulation comprising
   a. 0.5-5 wt. % nucleoside or nucleotide pro-drug, wherein the nucleoside or nucleotide pro-drug is a phosphoramidate phosphonamidate, phosphoester or sugar-ester nucleoside or nucleotide pro-drug
   b. 2-15 wt. % pyrrolidone-containing compound; and
   c. 65-97.5 wt. % of one or more co-solvent(s).
B. The ready-to-dilute formulation according to embodiment A, wherein the pyrrolidone containing compound is a pyrrolidone-containing polymer.
C. The ready-to-dilute formulation according to embodiment B, wherein the pyrrolidone-containing polymer is polyvinylpyrrolidone.
D. The ready-to-dilute formulation according to embodiment B or C, wherein the pyrrolidone-containing polymer has a number average molecule weight between 2000 and 70000 g/mol, more preferably between 2000 and 7000 g/mol.
E. The ready-to-dilute formulation of any preceding embodiment, wherein the one or more co-solvent(s) comprises one or more PEG co-solvent(s).
F. The ready-to-dilute formulation according to embodiment E, wherein the formulation comprises one or more PEG co-solvent(s) in an amount of 50-97.5 wt. %
G. The ready-to-dilute formulation of embodiment F, wherein the ready-to-dilute formulation comprises 20-50 wt. % PEG 300 and 20-50 wt. % PEG 400.
H. The ready-to-dilute formulation according to any preceding embodiment, wherein the one or more co-solvent(s) comprises propylene glycol in an amount up to about 25 wt. %
I. The ready-to-dilute formulation according to embodiment H, wherein the propylene glycol is present in an amount from 5 wt. % to 22 wt. %
J. The ready to dilute formulation according to any preceding claim, comprising one or more surfactant(s) in an amount of up to 15 wt. %
K. The ready-to-dilute formulation according to embodiment J, wherein the one or more surfactant(s) is present in an amount from 5 wt. % to 11 wt. %.
L. The ready-to-dilute formulation according to embodiments J-K, wherein the surfactant(s) is polysorbate.
M. The ready-to-dilute formulation according to embodiment L, wherein the polysorbate is polysorbate 80.
N. The ready-to-dilute formulation according to any preceding embodiments, comprising 1 wt. % to 3 wt. % nucleoside or nucleotide pro-drug
O. The ready-to-dilute formulation according to any preceding embodiments comprising 4 wt. % to 11 wt. % pyrrolidone-containing compound.
P. The ready-to-dilute formulation according to any preceding embodiment, wherein the remdesivir concentration is at least 12.5 mg/mL
Q. The ready-to-dilute formulation according to any preceding embodiment, wherein the remdesivir concentration is at least 15 mg/mL
R. The ready-to-dilute formulation according to any preceding embodiment, wherein the remdesivir concentration is at least 20 mg/mL
S. The ready-to-dilute formulation according to any preceding embodiment, wherein the ready-to-dilute formulation is free of a cyclodextrin compound or salts thereof.
T. The ready-to-dilute formulation according to any preceding embodiment, wherein the ready-to-dilute formulation is free of a cyclodextrin or salts thereof.
U. The ready-to-dilute formulation according to any preceding embodiment, wherein the ready-to-dilute formulation is free of buffers or pH modifiers.
V. The ready-to-dilute formulation according to any preceding embodiment, wherein the ready-to-dilute formulation is a ready-to-dilute solution.
W. The ready-to-dilute formulation according to any preceding embodiment, wherein nucleoside or nucleotide pro-drug is remdesivir.
X. The ready-to-dilute formulation according to embodiment W, wherein the ready-to-dilute formulation comprises:
   1-3 wt. % remdesivir
   4-8 wt. % polyvinylpyrrolidone, preferably polyvinylpyrrolidone K-12,
   65-80 wt. % PEG co-solvent(s)
   5-11 wt. % Polysorbate 80
Y. The ready-to-dilute formulation according to embodiment X, wherein the ready-to-dilute formulation comprises:
   25-35 wt. % PEG 300, and
   40-50 wt. % PEG 400
Z. The ready-to-dilute formulation according to embodiment X or Y, wherein the ready-to-dilute formulation comprises:
   5 to 15 wt. % propylene glycol
AA. An injectable solution comprising
   a. the ready-to-dilute formulation of embodiments A-Y, and
   b. an aqueous diluent.
BB. The injectable solution according to embodiment AA, wherein the aqueous diluent comprises water, NaCl solution and/or dextrose solution.
CC. The injectable solution according to embodiment AA, wherein the aqueous diluent is water.
DD. The injectable solution according to any one of embodiments AA-CC, wherein the ratio of ready-to-dilute solution to aqueous diluent is 1:8 to 1:100, more preferably from 1:8 to 1:18
EE. The injectable solution according to any one of embodiments AA-DD, wherein the osmolality of the injectable solution is 300±50 mOsm
FF. The injectable solution according to any one of embodiments AA-EE, wherein the pH of the injectable solution is between 4 and 8, more preferably between 5 and 7.
GG. A method of treating a viral infection, the method comprising intravenously administering to a subject in need thereof the injectable solution of embodiments AA-DD
HH. A method of treating a viral infection according to embodiment EE, wherein the virus causing the viral infection is selected from a coronavirus, respiratory syncytial virus, ebola, hepatitis, junin, lassa fever, orthomyxovirus, Hepatitis Virus (HV) type, disease-causing picornavirus, Ebola, SARS, MERS, respiratory syncytial virus and other pneumovirus, influenza, polio measles and retrovirus including adult Human T- cell lymphotropic virus type 1 (HTLV-1) and human immunodeficiency virus (HIV).
II. A method of treating a viral infection according to embodiment FF, wherein the viral infection is a coronavirus
JJ. A method of treating a viral infection according to embodiment GG, wherein the viral infection is SARS-COV-2

### EXAMPLES

### Initial Screening-Effect of Various Excipients on Solubility

Various excipients were first screened for their solubilizing effect on remdesivir, with results shown in Table 1.

TABLE 1
Effect of Excipients on remdesivir solubility

| Excipient | Solubility (mg/mL) |
|---|---|
| N-methyl-2-pyrrolidone (NMP) | ≥112 |
| Soluplus ® + Ethanol + Water | ≥34 |
| Plasdone (PVP) + PEG 300 | ≥66 |
| NMP + Ethanol | ≥226 |
| Ethanol | ≥21 |
| Benzyl Alcohol | ≥56 |
| Propylene Glycol | ≥29 |
| PEG 300 | ≥39 |
| PEG 400 | ≥16 |
| PEG 600 | ≥6.2 |
| PEG 1000 | ≥6.3 |
| PEG 400 + Glycerol (3:5) | 0.011 |
| PEG 400 + Glycerol (3:10) | 0.0008 |
| Castor Oil | 0 |
| Sesame Oil | 0 |
| Peppermint Oil | 15.9 |
| Soybean Oil | 0 |
| Peanut Oil | — |
| Mineral Oil | — |
| Span 80 | 0.3 |
| Span 20 | 0.006 |

It was observed that remdesivir has a high solubility (over 50 mg/ml) in low molecular weight polyethylene glycols (PEG 300 and 400) in the presence of low molecular weight polyvinylpyrrolidone compounds (PVP or povidone, K-25, K30, K-12, K-17, etc). Such solubilities were notably higher in PVP and PEG as compared to pure PEG. Solubility in N-methyl pyrrolidone, a liquid, was also especially high (>100 mg/mL).

### Effect of Surfactants on Solubility

The effect of various surfactants on the solubility of remdesivir in water was also tested. The results are shown in Table 2.

TABLE 2
Examples of solubility of remdesivir in water in the presence
of various surfactants and at different concentrations

| | | Concentration | Remdesivir |
|---|---|---|---|
| | HLB | in Water | Solubility |
| Surfactant | value | (% w/w) | (mg/mL) |
| TWEEN 20 (Polysorbate 20) | 16.7 | 0.16 | 0.016 |
| TWEEN 80 (Polysorbate 80) | ~15 | 0.30 | 0.033 |
| Poloxamer P188 | ~29 | 0.07 | 0.000 |
| Poloxamer P407 | ~18 | 0.07 | 0.006 |
| Kolliphor EL (Polyoxyl 35 | ~15 | 0.10 | 0.020 |
| Castor Oil; Cremophor) | | | |

The above results indicate that the solubility of remdesivir can be increased in the presence of cosolvents and solubilizing excipients and surfactants. The data also indicates that by using certain combinations of cosolvents and solubilizing excipients for an injectable dosage form that does not contain SBEBCD (as compared to commercially available Veklury® formulations), it can be possible to achieve high concentrations of remdesivir in a ready-to-dilute solution that can also be diluted in an aqueous media without causing the drug to precipitate. In addition to achieving high concentration of the active ingredient in the ready-to-dilute solution, it is important to maintain sufficient solubility in the final ready-to-administer formulation following dilution with an aqueous infusion medium to deliver adequate doses of remdesivir in the form of an intravenous infusion.

### Example Ready-to-Dilute Formulations

Based on the data above, various ready-to-dilute formulations were prepared. It was found that formulations comprising PEG and PVP could effectively sustain the solubility of remdesivir in solution even when diluted with an aqueous diluent (e.g. to form a ready-to-administer injectable solution). Unexpectedly, this combination of components also led to remdesivir having excellent chemical stability. This is surprising considering remdesivir is a nucleotide pro-drug, which are prone to degradation and hydrolysis.

TABLE 3
Example Formulations

| | | Batch Numbers | | | | |
|---|---|---|---|---|---|---|
| | | ES040- | ES040- | ES040- | ES040- | ES040- |
| Component | Function | 152 | 168A | 168B | 187C | 190 |
| | | % (w/w) | | | | |
| Remdesivir | Active | 2.13 | 2.13 | 2.35 | 1.28 | 1.40 |
| | Ingredient | | | | | |
| Povidone | Pyrrolidone | 9.49 | 9.69 | 10.40 | 5.83 | 5.83 |
| (PVP) | Compound/ | | | | | |
| K-12 | Solubilizing | | | | | |
| | Aid | | | | | |
| PEG 300 | Cosolvent | 40.55 | 40.63 | 44.68 | 28.94 | 28.91 |
| PEG 400 | Cosolvent | 39.34 | 39.07 | 21.28 | 46.71 | 46.65 |
| Polysorbate | Surfactant | — | — | — | 8.16 | 8.15 |
| 80 | | | | | | |
| (Tween 80) | | | | | | |
| Propylene | Cosolvent | 8.49 | 8.49 | 21.29 | 9.08 | 9.07 |
| Glycol | | | | | | |

The components of ready-to-dilute formulations were found to effectively sustain the solubility of remdesivir in solution once diluted with an aqueous based diluent, (e.g., by simply combining with water-for-injection (WFI), or by combining with an aqueous diluent comprising one or more surfactants). The solubility of remdesivir in the injectable solution was even further improved by the presence of a surfactant, e.g., in the case of ES040-187C and ES040-190 where a polysorbate 80 (i.e., Tween 80) surfactant is used.

In addition to increased solubility, it was observed that remdesivir has excellent chemical stability when solubilized with both PVP and PEG. This was surprising because in other non-aqueous solutions (e.g. including formulations comprising PEG in the absence of PVP), remdesivir was found to be prone to hydrolysis.

In comparison, the ready-to-dilute formulations of the present invention showed excellent stability of remdesivir at ambient temperature and when stored over time at elevated temperatures (40° C.). Stability was assessed by a stability indicating HPLC method that tracked the peak of the active ingredient, remdesivir, and all related substances (RS) formed by the degradation of remdesivir. The peak area (mAU*s) of remdesivir at any time point was divided by the total peak area of remdesivir and its related substances formed due to degradation during manufacturing and storage, and the ratio was expressed as a percentage of remdesivir remaining in the solution at that time point. The results are shown in Table 4.

TABLE 4
Relative peak area over time at 40° C.

| For- | | | | | | | |
|---|---|---|---|---|---|---|---|
| mulation | | | | | | | |
| (Batch | | | | | | | |
| No.) | t₀ | t _{2 weeks} | t _{1 month} | t _{2 months} | t _{3 months} | t _{6 months} | t _{7 months} |
| ES040- | 100% | 99% | 100% | | 100% | | 99.2% |
| 168A | | | | | | | |
| (25 mg/ | | | | | | | |
| mL) | | | | | | | |
| ES040- | 100% | 100% | 99% | 99% | 100% | 99.5% | |
| 190 | | | | | | | |
| (15 mg/ | | | | | | | |
| mL) | | | | | | | |

Furthermore, due to the excellent stability of remdesivir in the ready-to-dilute formulation, no substantial degradation or hydrolysis of remdesivir was observed even after sterilization with steam at 121° C. for 30 minutes. Solutions of remdesivir in PEG+PVP mixtures were all very stable with recoveries over 98% compared to that of the presterilized solution (Table 5). Recovery was assessed by a HPLC assay method by first comparing the area under the peak for remdesivir in a freshly prepared standard solution of the drug to that of the peak area of remdesivir in the test sample to calculate the concentration of the drug remaining in the test sample and, then, dividing this measured concentration of remdesivir at any given time point with that in the freshly prepared formulation (to) and expressing this ratio as a percentage of drug remaining compared to the initial (to) . . . . Due to several dilution steps and rounding of numbers, a ±2% variability were considered acceptable in the measured Recovery results.

TABLE 5
Recovery of remdesivir in example Formulations
heated to 121° C. for 30 minutes.

| | Concentration of | Concentration of | |
|---|---|---|---|
| Formulation | Remdesivir Before | Remdesivir After | |
| (Batch No.) | Sterilization | Sterilization | % Recovery |
| ES040-152 | 23.8 mg/mL | 23.4 mg/mL | 98.3% |
| ES040-168A | 23.6 mg/mL | 23.2 mg/mL | 98.4% |
| ES040-168B | 25.4 mg/mL | 25.8 mg/mL | 101.3% |
| ES040-187C | 13.67 mg/mL | 13.45 mg/mL | 98.4% |

### Properties of Ready-To-Dilute and Injectable Solutions

The components of the ready-to-dilute formulations are free of sulfobutylether β-cyclodextrin (SBEBCD). In addition, the quantity and concentration of each inactive ingredient in the formulation is such that their maximum daily intake when the maximum daily dose of the drug (remdesivir in the example) is administered does not exceed the maximum daily exposure from other approved products, and/or as listed in FDA's inactive ingredients database (IID) (Table 6).

TABLE 6
ES040-190 as an Example Formulation
for the Ready-to-dilute Solution

| | | Max | FDA's IIG |
|---|---|---|---|
| | % | Daily | Database Maximum |
| Component | (w/w) | Intake (g) | Daily Exposure (MDE) |
| Remdesivir | 1.40 | 0.200 | |
| PVP K-12 | 5.83 | 0.832 | 1.000 |
| (2-Pyrrolidone K-12) | | | |
| PEG 300 | 28.91 | 4.128 | ≤4.875 (based on |
| | | | approved product) |
| PEG 400 | 46.65 | 6.663 | 7.470 |
| Polysorbate 80 | 8.15 | 1.164 | 4.680 |
| (Tween 80) | | | |
| Propylene Glycol | 9.07 | 1.295 | 16.624 |
| Total | 100 | | |
| WFI (Following | | | |
| Dilution) | | | |

When Formulation ES040-190 that is listed as an example in Table 6 is diluted with 100 ml of water-for-injection (WFI), the final diluted solution contains over 92% water and is isotonic with (300±25 mOsm) with blood. Alternatively, where necessary, the product can also be diluted in 250 mL of half normal saline (0.45% sodium chloride for infusion) or 250 ml of 5% dextrose solution to provide a solution isotonic with blood, thereby providing flexibility of administration methods over Veklury®.

The novel product is a clear, colorless to slightly yellow, nonaqueous sterile solution filled into a clear, type-I glass vial with a rubber stopper and aluminium flip-off crimp-cap. When mixed with water or various aqueous infusion media, the product readily dissolves at all concentrations to result in a clear colorless solution after dilution. Due to the higher concentration of the drug in the concentrated solution and relatively low dose-volume (≤7 mL for 100 mg remdesivir as compared to 20 mL for Veklury®), it is not necessary to withdraw and replace this added volume from the IV infusion fluid bag/bottles unlike Veklury®. The entire dose volume can simply be added to a container containing sterile WFI (or other) bag/bottle, and then mixed and injected. More specifically and as conceived, the entire dose-volume can be contained in the glass vial and can be withdrawn using an aseptic needle and syringe assembly and injected into the infusion vial or bag and mixed, before administration.

Alternatively, the concentrated solution may be filled into special vials of the type similar to Add-Vantage™ vials by Pfizer (or other such vials by other manufacturers), where the contents of the vial can be directly mixed with the IV infusion fluid in a bag through a special adaptor connecting the vial and infusion bag, without requiring the medication to be withdrawn by a syringe+needle assembly and re-injected into the bag/bottle. As yet another alternative, the product can be filled into two-chamber bags or bottles, where the concentrated solution may be separated from the infusion fluid by a temporary barrier that can be non-intrusively removed for the contents to be mixed prior to administration by IV infusion. For consistency of use, therapeutic equivalency and ease of switching to the improved product, the final administration method can be designed to match that of the reference drug, Veklury®.

TABLE 7
Comparison of Novel Formulation as compared With Veklury ® formulations

| | | | Emphascience's Novel |
|---|---|---|---|
| | Veklury ® for | | Injection Formulation (based |
| | Injection | Veklury ® Injection | on ES040-190 |
| Active Ingredient | Remdesivir | Remdesivir | Remdesivir |
| Dosage Form | Lyophilized powder | Concentrated ready- | Concentrated ready-to-dilute |
| | | to-dilute solution | solution (100 mg/6.7 mL) |
| | | (100 mg/20 mL) | |
| Ready-to-Dilute | Contains | Contains | Nonaqueous solution. |
| Formulation | sulfobutylether | sulfobutylether beta- | Contains polyethylene glycol |
| | beta-cyclodextrin | cyclodextrin for | 300 and 400, and propylene |
| | for complexation | complexation and | glycol as co-solvents, and |
| | and solubilization of | solubilization of drug; | polyvinylpyrrolidone (K12) and |
| | drug; 3 g SBEBCD | 6 g SBEBCD for | Tween 80 as solubilizers. |
| | for every 100 mg | every 100 mg | |
| | remdesivir | remdesivir | |
| Storage | 20-25° C. (Controlled | 2-8° C. (Refrigerated) | 20-25° C. (Projected) |
| Temperature | RT) | | |
| pH | ca. 3.5 (after | ca. 3.5 | 5 to 7 (same pH as IV infusion |
| | reconstitution) | | medium used for final dilution). |

In another example, the concentrated, ready-to-dilute solution ES040-168B is diluted with a unique diluent, ES040-170D at a pH of 4.4comprising 97.6% water, 0.36% polysorbate and 2.05% PEG400. The composition of the final mixture, following dilution to a remdesivir concentration of 1 mg/mL, will be as listed in Table 8.

TABLE 8
Composition of final diluted solution prepared by mixing a unique
diluent (ES040-170D) comprising 97.6% water, 0.36% polysorbate 80
and 2.05% polyethylene glycol 400 with the concentrated ready-to-dilute
solution, ES040-168B (see Table 3 for composition) containing 25 mg/mL
of remdesivir to a finalconcentration of 1 mg/ml ofremdesivir.
Diluted solution

| | % w/w |
|---|---|
| Remdesivir | 0.1 |
| Povidone K12 | 0.455 |
| PEG 300 | 1.909 |
| PEG 400 | 3.884 |
| Propylene glycol | 0.399 |
| Polysorbate8 80 | 0.36 |
| Water for injection | 97.596 |

### Comparative Examples

Various solutions formulations of Remdesivir with and without polyvinylpyrrolidone and sulfobutylether cyclodextrin were tested for their stability.

As can be seen from the above data, ES040-190 formulation showed superior stability over time at 40 degrees as compared to other formulations not containing polyvinylpyrrolidone, including over formulations comprising sulfobutylether cyclodextrin (i.e., mimicking the current Veklury® formulations).

TABLE 10
Stressed stability at 40° C. of Ready-to-dilute Formulations with
(ES040-148) and without (ES040-190) SBEBCD. ES040-148 matches
the marketed formulation for Veklury ® Injection and
ES040-190 is the novel ready-to-dilute solution formulation.

| | Relative Peak Area of Remdesivir (%) | | | | | |
|---|---|---|---|---|---|---|
| | t₀ | 2 weeks | 1 month | 2 months | 3 months | 6 months |
| ES040-148^{†i} | 100% | 97.2% | 94% | Not analyzed. Study | | |
| | | | | terminated due to | | |
| | | | | significant degradation. | | |
| ES040-190ⁱⁱ | 100% | 100% | 99% | 99% | 100% | 99.5% |

ⁱFormulation mimicking marketed Veklury ® Injection containing SBEBCD, but no polyvinylpyrrolidone
ⁱⁱNovel injection concentrate containing povidone and 15 mg/mL remdesivir, comprising polyvinylpyrrolidone, formulation is as described above.
^{†}Denotes Comparative Example

### Summary

In summary, the novel product offers many advantages over Veklury®. The significantly higher stability of remdesivir in the formulation and high solubility without the use of cyclodextrins are key advantages and can improve compliance, safety and therapeutic outreach. The novel product is stable and can be expected to have an extended shelf-life at room temperature longer than that for Veklury® Injection and comparable to that of Veklury® for Injection (the lyophilized powder). The novel product can be manufactured and stored under ambient conditions without special environmental controls (such as low UV light or low oxygen). The novel product is also stable when subjected to steam-sterilization, thus providing an easy and reliable method for sterilization for maximum sterility assurance. The lower fill-volume (≤7 mL instead of 20 mL for Veklury® Injection) allows for faster filling into commonly available and smaller vial sizes that lower material, manufacturing and shipping costs. The higher solubility of remdesivir in the novel formulation composition combined with its improved stability are especially unique advantages that could enable usage under conditions where refrigeration and aseptic handling are not readily available. These highly stable, water-miscible solution formulations containing high concentrations of remdesivir as described in Table 3 can be used to design other dosage forms, applications where the final concentration and dose-volume can also differ from that in Veklury®. Since the concentration of remdesivir in the novel formulation can be higher than 15 mg/mL and ≥25 mg/mL, the formulation can also be used to administer therapeutically effective doses by intranasal, oral and various routes of administration in addition to the IV infusion. For example, the same stable, concentrated solution formulation or one utilizing a similar stabilizing and solubilizing strategy with povidone and PEG along with added flavors can be used as an oral solution dosage form.

1. A ready-to-dilute formulation comprising
5-5 wt. % of a nucleoside or nucleotide pro-drug, the nucleoside or nucleotide pro-drug being a phosphoramidate, phosphonamidate, phosphoester or sugar-ester nucleoside or nucleotide pro-drug
2-15 wt. % pyrrolidone-containing compound; and
65-97.5 wt. % of one or more co-solvent(s).

2. The ready-to-dilute formulation according to claim 1, wherein the pyrrolidone-containing compound is a pyrrolidone-containing polymer.

3. The ready-to-dilute formulation according to claim 2, wherein the pyrrolidone-containing polymer is polyvinylpyrrolidone.

4. The ready-to-dilute formulation according to claim 2, wherein the pyrrolidone-containing polymer has a weight average molecule weight between 2000 and 70000 g/mol.

5. The ready-to-dilute formulation of claim 1, wherein the one or more co-solvent(s) comprises one or more PEG co-solvent(s).

6. The ready-to-dilute formulation according to claim 5, wherein the formulation comprises one or more PEG co-solvent(s) in an amount of 50-97.5 wt. %

7. The ready-to-dilute formulation of claim 6, wherein the ready-to-dilute formulation comprises 20-50 wt. % PEG 300 and 20-50 wt. % PEG 400.

8. The ready-to-dilute formulation according to claim 1, wherein the one or more co-solvent(s) comprises propylene glycol in an amount up to about 25 wt. %

9. The ready to dilute formulation according to claim 1, further comprising one or more surfactant(s) in an amount of up to 15 wt. %

10. The ready-to-dilute formulation according to claim 9, wherein the one or more surfactant(s) is a polysorbate.

11. The ready-to-dilute formulation according to claim 10, wherein the polysorbate is polysorbate 80.

12. The ready-to-dilute formulation according to claim 1, wherein the formulation comprises 1 wt. % to 3 wt. % nucleoside or nucleotide pro-drug.

13. The ready-to-dilute formulation according to claim 1, wherein the formulation comprises 4 wt. % to 11 wt. % pyrrolidone-containing compound.

14. The ready-to-dilute formulation according to claim 1, wherein the formulation comprises remdesivir at a concentration of at least 12.5 mg/ml

15. The ready-to-dilute formulation according to claim 1, wherein the ready-to-dilute formulation is free of a cyclodextrin compound or salts thereof.

16. The ready-to-dilute formulation according to claim 1, preceding claim, wherein the nucleoside or nucleotide pro-drug is remdesivir.

17. An injectable solution comprising
the ready-to-dilute formulation of claim 1, and
an aqueous diluent.

18. The injectable solution according to claim 17, wherein the aqueous diluent comprises water, NaCl solution and/or dextrose solution and/or salts, buffers, pH and/or tonicity adjusters.

19. The injectable solution according to claim 17, wherein the ratio of ready-to-dilute solution to aqueous diluent is 1:8 to 1:100.

20. A method of treating a viral infection, the method comprising intravenously administering to a subject in need thereof the injectable solution of claim 17.

21. The method according to claim 20, wherein the virus causing the viral infection is selected from a coronavirus, respiratory syncytial virus, ebola, hepatitis, junin, lassa fever, orthomyxovirus, Hepatitis Virus (HV) type, disease-causing picornavirus, Ebola, SARS, MERS, respiratory syncytial virus and other pneumovirus, influenza, polio measles and retrovirus including adult Human T-cell lymphotropic virus type 1 (HTLV-1) and human immunodeficiency virus (HIV).
